SEQUENCE LISTING (30 sequences)

SEQ ID NO: 1 - protein, 114 aa, synthetic construct
EVQLVESGGGLVQPGGSLRLSCAASGSIFSPNGIGWYRQAPGKLRELVAGITGNGLTDYAESVKGRFTISRDNSKNTVYLQMNSLRAEDTAVYYCNLLLNHAEWGQGTLVTVSS

SEQ ID NO: 2 - protein, 114 aa, synthetic construct
EVQLVESGGGLVQPGGSLRLSCAASGIRFSIANMGWYRQAPGKLHEMVAGITSDGFTNYADSVKGRFTISRDNSKNTLYLQMNSLRAEDTAVYYCASFPTRHYWGQGTLVTVSS

SEQ ID NO: 3 - protein, 119 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAA

SEQ ID NO: 4 - protein, 108 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKR

SEQ ID NO: 5 - protein, 330 aa, synthetic construct [sequence omitted]

SEQ ID NO: 6 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 107 aa, synthetic construct [sequence omitted]

SEQ ID NO: 8 - protein, 19 aa, synthetic construct
MGWSCIILFLVATATGVHS

SEQ ID NO: 9 - protein, 5 aa, synthetic construct
GGGGS

SEQ ID NO: 10 - protein, 448 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHENPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 574 aa, synthetic construct
EVQLVESGGGLVQPGGSLRLSCAASGIRFSIANMGWYRQAPGKLHEMVAGITSDGFTNYADSVKGRFTISRDNSKNTLYLQMNSLRAEDTAVYYCASFPTRHYWGQGTLVTVSSGGGGSGGGGSGGGGSQVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 577 aa, synthetic construct
EVQLVESGGGLVQPGGSLRLSCAASGIRFSIANMGWYRQAPGKLHEMVAGITSDGFTNYADSVKGRFTISRDNSKNTLYLQMNSLRAEDTAVYYCASFPTRHYWGQGTLVTVSSGGGGSGGGGSGGGGSQVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 577 aa, synthetic construct
EVQLVESGGGLVQPGGSLRLSCAASGSIFSPNGIGWYRQAPGKLRELVAGITGNGLTDYAESVKGRFTISRDNSKNTVYLQMNSLRAEDTAVYYCNLLLNHAEWGQGTLVTVSSGGGGSGGGGSGGGGSQVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 577 aa, synthetic construct; identical to SEQ ID NO: 14

SEQ ID NO: 16 - protein, 577 aa, synthetic construct; identical to SEQ ID NO: 14

SEQ ID NO: 17 - protein, 574 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASGSIFSPNGIGWYRQAPGKLRELVAGITGNGLTDYAESVKGRFTISRDNSKNTVYLQMNSLRAEDTAVYYCNLLLNHAEWGQGTLVTVSS

SEQ ID NO: 18 - protein, 344 aa, synthetic construct
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 5 aa, synthetic construct
NYDIN

SEQ ID NO: 20 - protein, 17 aa, synthetic construct
WIFPGDGSTQYNEKFKG

SEQ ID NO: 21 - protein, 9 aa, synthetic construct
QTTATWFAY

SEQ ID NO: 22 - protein, 11 aa, synthetic construct
RASQSISDYLH

SEQ ID NO: 23 - protein, 7 aa, synthetic construct
YASQSIS

SEQ ID NO: 24 - protein, 8 aa, synthetic construct
QNGHSFPL

SEQ ID NO: 25 - protein, 10 aa, synthetic construct
GITFSRSVMG

SEQ ID NO: 26 - protein, 10 aa, synthetic construct
GIRFSIANMG

SEQ ID NO: 27 - protein, 10 aa, synthetic construct
GSIFSPNGIG

SEQ ID NO: 28 - protein, 10 aa, synthetic construct
GSIFSPNAIG

SEQ ID NO: 29 - protein, 10 aa, synthetic construct
GSIFSPQGIG

SEQ ID NO: 30 - protein, 16 aa, synthetic construct
GITSDDNTDYADSVKG

(19) United States
(12) Patent Application Publication
(10) Pub. No.: US 2025/0092140 A1
(43) Pub. Date: Mar. 20, 2025

(54) BISPECIFIC ANTIBODY BINDING TO B7H3 AND NKP30, AND APPLICATION THEREOF

(71) Applicant: SHENGHE (CHINA) BIOPHARMACEUTICAL CO., LTD., Nanjing, Jiangsu (CN)

(72) Inventors: Chong ZHOU, Nanjing (CN); Xiaoling JIANG, Nanjing (CN); Liusong YIN, Nanjing (CN)

(73) Assignee: SHENGHE (CHINA) BIOPHARMACEUTICAL CO., LTD., Nanjing, Jiangsu (CN)

(21) Appl. No.: 18/727,795

(22) PCT Filed: Jan. 12, 2023
(86) PCT No.: PCT/CN2023/071850

§ 371 Date: Jul. 10, 2024

(30) Foreign Application Priority Data
Jan. 14, 2022 (WO) PCT/CN2022/071902

(51) Int. Cl. C07K 16/28

(52) U.S. Cl. CPC C07K 16/2827; C07K 16/2803; C07K 2317/31; C07K 2317/569; C07K 2317/73; C07K 2317/92

(57) ABSTRACT

In the technical field of biomedicine, a bispecific antibody including (a) a first antibody that specifically binds to a first antigen or antigen binding fragment thereof; and (b) a second antibody that specifically binds to a second antigen or antigen binding fragment thereof. Specifically, the first antigen is B7H3 and the second antigen is NKP30, or the first antigen is NKP30 and the second antigen is B7H3.

### TECHNICAL FIELD

The present invention belongs to the technical field of biomedicine, and relates to a bispecific antibody binding B7H3 and NKP30 and application thereof.

### BACKGROUND

B7H3 (CD276) was identified as a member of the B7 family, and TLT-2 was identified as its potential receptor in 2001. Recent studies have shown that B7H3 is both an immune co-stimulatory and co-inhibitory molecule, and has anti-tumor activity and can trigger immune escape. Therefore, its tumor-associated antigen properties are mostly used in antibody drug development to kill tumor cells with high expression of B7H3 through ADC drugs, ADCC mechanisms, etc.

Northern blot analysis showed that B7H3 mRNA was widely expressed in a variety of normal tissues including liver, small intestine, pancreas, testis, heart, colon, etc, but not expressed in human peripheral blood leukocytes. However, B7H3 protein is only expressed at low levels, but its expression can be induced in B cells, T cells, monocytes or NK cells by granulocyte-macrophage colony-stimulating factor (GM-CSF) or lipopolysaccharide (LPS) stimulation.

Soluble B7H3 (sB7H3) has been shown to be released by monocytes, dendritic cells (DCs) and activated T cells. High expression of B7H3 was negatively correlated with tumor size in breast cancer patients. B7H3 was expressed in 93% of ovarian tumors compared to normal ovarian tissue, and B7H3 expression was associated with staging, high recurrence and low survival rates in ovarian tumor. B7H3 expression was also significantly elevated in colorectal cancer and may be involved in the development of colorectal cancer. In addition, B7H3 protein is also expressed in prostate cancer, pancreatic cancer, squamous cell carcinoma, non-small cell lung cancer and gastric cancer. The aberrant expression of B7H3 in many tumors suggests that B7H3 may be a useful marker for the study of tumorigenesis, progression, diagnosis and treatment.

Therefore, we can target the physiological functions of B7H3 in tumor growth, migration and invasion to develop antibody drugs that use ADC drugs, ADCC mechanism, etc. to kill tumor cells that express high levels of B7H3 in the tumor microenvironment.

Human NKP30 (Natural cytotoxicity triggering receptor 3) protein, encoded by the NCR3 gene, is a member of the natural cytotoxicity triggering receptors (NCRs) family, which are activation receptor on the surface of NK cells.

NK cells are the main “fighters” in the body responsible for killing abnormal cells such as aged, virus-infected and tumor cells. NKP30 is expressed in all resting and activated NK cells, multiple effector NKT cells, γδ T-cells and MAIT cells, and can activate NK cells, γδ T-cells and other tumor-killing cells to kill tumors.

NKP30 is a major trigger receptor for killing certain tumors, such as melanoma MEL15. NKP30 can also synergise with other activation receptors to enhance the activity of NK cells. Therefore, the development of bispecific antibodies targeting tumor-associated antigens and NKP30, bridging tumor cells to NK cells and activating only NK cells in the tumor microenvironment, may help to reduce side effects, avoid the high activation of systemic NK cells, and reduce the abnormal cell sensitivity to the loss of MHC molecules.

### SUMMARY

The present invention provides a bispecific antibody comprising:
(a) a first antibody or antigen binding fragment thereof that specifically binds to a first antigen; and
(b) a second antibody or antigen binding fragment thereof that specifically binds to a second antigen;
wherein, the first antigen is B7H3 and the second antigen is NKP30; or, the first antigen is NKP30 and the second antigen is B7H3.

In alternative embodiments, the first antibody or antigen binding fragment thereof comprises a heavy chain and a light chain, and the second antibody or antigen binding fragment thereof comprises VHH;
wherein, the VHH is linked to the N-terminal or C-terminal end of the heavy or the light chain of the first antibody or antigen binding fragment thereof.

In alternative embodiments, the heavy chain variable region of one heavy chain of the first antibody or antigen binding fragment thereof forms an antigen binding site with one light chain variable region of the light chain, and the heavy chain variable region of the other heavy chain forms an antigen binding site with the light chain variable region of the other light chain.

In alternative embodiments, the bispecific antibody comprises one first antibody or antigen binding fragment thereof and one or more of VHH(s).

In alternative embodiments, the bispecific antibody comprises one first antibody or antigen binding fragment thereof and one VHH, wherein the VHH is linked to the N-terminal or C-terminal end of the heavy or the light chain of the first antibody or antigen binding fragment thereof.

In alternative embodiments, the bispecific antibody comprises one first antibody or antigen binding fragment thereof and two VHHs.

In alternative embodiments, two VHHs are linked to the N-terminal end of each of the two heavy or two light chains of the first antibody or antigen binding fragment thereof; or, two VHHs are linked to the C-terminal end of each of the two heavy or two light chains of the first antibody or antigen binding fragment thereof.

In alternative embodiments, the bispecific antibody comprises two first polypeptide chains and two second polypeptide chains, wherein,
(a) the first polypeptide chain each independently comprises a heavy chain of the first antibody or antigen binding fragment thereof and the VHHs; and
(b) the second polypeptide chain each independently comprises a light chain of the first antibody or antigen binding fragment thereof.

In alternative embodiments, the bispecific antibody comprises two first polypeptide chains and two second polypeptide chains, wherein,
(a) the first polypeptide chain each independently comprises a heavy chain of the first antibody or antigen binding fragment thereof; and
(b) the second polypeptide chain each independently comprises a light chain of the first antibody or antigen binding fragment thereof and the VHHs.

In alternative embodiments, two first polypeptide chains are the same or different, and/or two second polypeptide chains are the same or different.

In alternative embodiments, two VHHs are linked via a linker to the N-terminal or C-terminal ends of the two heavy chains or the two light chains of the first antibody or antigen binding fragment thereof, respectively.

In alternative embodiments, the linker has an amino acid sequence as shown by (G₄S)x, wherein x is an integer selected from 1-6; preferably, x is an integer selected from 1-3.

In alternative embodiments, the heavy chain of the first antibody or antigen binding fragment thereof comprises a heavy chain variable region and a heavy chain constant region, and the light chain comprises a light chain variable region and a light chain constant region; preferably, the first antibody or antigen binding fragment thereof is a full-length antibody.

In alternative embodiments, the heavy chain of the first antibody or antigen binding fragment thereof comprises a first Fc region and a second Fc region.

In alternative embodiments, the first Fc region and the second Fc region are the same or different.

In alternative embodiments, the Fc regions are selected from the group consisting of: IgG, IgA, IgD, IgE and/or IgM.

In alternative embodiments, the Fc regions are selected from the group consisting of: IgG1, IgG2, IgG3 and/or IgG4.

In alternative embodiments, the Fc regions are selected from the group consisting of: mouse IgG1, IgG2a, IgG2b and/or IgG3, or from rat IgG1, IgG2a, IgG2b and/or IgG2c.

In alternative embodiments, the Fc regions are selected from the group consisting of: IgG1, IgG2, IgG3 and/or IgG4.

In alternative embodiments, the first antibody or antigen binding fragment thereof specifically binds to B7H3, and the VHH specifically binds to NKP30, wherein,
the first antibody or antigen binding fragment thereof comprises HCDR1 as shown by SEQ ID NO: 19, HCDR2 as shown by SEQ ID NO: 20, and HCDR3 as shown by SEQ ID NO: 21; and LCDR1 as shown by SEQ ID NO: 22, LCDR2 as shown by SEQ ID NO: 23, and LCDR3 as shown by SEQ ID NO: 24.

In alternative embodiments, the amino acid sequence of the heavy chain variable region VH of the first antibody or antigen binding fragment thereof is as shown by SEQ ID NO: 3, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO: 3.

In alternative embodiments, the amino acid sequence of the light chain variable region VL of the first antibody or antigen binding fragment thereof is as shown by SEQ ID NO: 4, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO: 4.

In alternative embodiments, the amino acid sequence of the IgG1 heavy chain constant region of the first antibody or antigen binding fragment thereof is as shown by SEQ ID NO: 5, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO: 5.

In alternative embodiments, the amino acid sequence of the IgG4 heavy chain constant region of the first antibody or antigen binding fragment thereof is as shown by SEQ ID NO: 6, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO: 6.

In alternative embodiments, the amino acid sequence of the light chain constant region of the first antibody or antigen binding fragment thereof is as shown by SEQ ID NO: 7, or is an amino acid sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO: 7.

In alternative embodiments, the first antibody or antigen binding fragment thereof is a mouse-derived antibody, a chimeric antibody, a humanized antibody or a fully human antibody.

In alternative embodiments, HCDR1 of the VHH is selected from any of the amino acid sequences of SEQ ID NO: 25 or SEQ ID NO: 28, or is a sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to any of the amino acid sequences of SEQ ID NO: 25 or SEQ ID NO: 28; HCDR2 is selected from any of the amino acid sequences of SEQ ID NO: 26 or SEQ ID NO: 29, or is a sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to any of the amino acid sequences of SEQ ID NO: 26 or SEQ ID NO: 29; HCDR3 is selected from any of the amino acid sequences of SEQ ID NO: 27 or SEQ ID NO: 30, or is a sequence having at least 80% identity to any of the amino acid sequences of SEQ ID NO: 27 or SEQ ID NO: 30.

In alternative embodiments, the heavy chain variable region VH of the VHH is selected from any of the amino acid sequences of SEQ ID NO: 1 or SEQ ID NO: 2, or is a sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to any of the amino acid sequences of SEQ ID NO: 1 or SEQ ID NO: 2.

In alternative embodiments, the VHH is a camel-derived antibody, a chimeric antibody, a humanized antibody or a fully human antibody.

In alternative embodiments, the first antibody or antigen binding fragment thereof comprises:
a heavy chain variable region VH as shown by SEQ ID NO: 3, and a light chain variable region VL as shown by SEQ ID NO: 4,
and, the VHH comprises:
(a) a heavy chain variable region VH as shown by SEQ ID NO: 1, or
(b) a heavy chain variable region VH as shown by SEQ ID NO: 2.

In alternative embodiments, the first polypeptide chain of the bispecific antibody is selected from any of the amino acid sequences of SEQ ID NO: 12, SEQ ID NO: 13, SEQ ID NO: 14, SEQ ID NO: 15, SEQ ID NO: 16 or SEQ ID NO: 17, or is a sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to any of the amino acid sequences of SEQ ID NO: 12, SEQ ID NO: 13, SEQ ID NO: 14, SEQ ID NO: 15, SEQ ID NO: 16 or SEQ ID NO: 17; and the second polypeptide chain of the bispecific antibody is as shown by SEQ ID NO: 11, or is a sequence having at least 80% (such as 80%, 81%, 82%, 83%, 84%, 85%, 86%, 87%, 88%, 89%, 90%, 91%, 92%, 93%, 94%, 95%, 96%, 97%, 98%, 99% or 100%) identity to SEQ ID NO: 11.

In alternative embodiments, the bispecific antibody comprises:
(1) the first polypeptide chain as shown by SEQ ID NO: 12, and the second polypeptide chain as shown by SEQ ID NO: 11;
(2) the first polypeptide chain as shown by SEQ ID NO: 13, and the second polypeptide chain as shown by SEQ ID NO: 11;
(3) the first polypeptide chain as shown by SEQ ID NO: 14, and the second polypeptide chain as shown by SEQ ID NO: 11;
(4) the first polypeptide chain as shown by SEQ ID NO: 15, and the second polypeptide chain as shown by SEQ ID NO: 11;
(5) the first polypeptide chain as shown by SEQ ID NO: 16, and the second polypeptide chain as shown by SEQ ID NO: 11; or
(6) the first polypeptide chain as shown by SEQ ID NO: 17, and the second polypeptide chain as shown by SEQ ID NO: 11.

The present invention also provides a nucleic acid encoding the bispecific antibody as described above.

The present invention also provides a vector or a plasmid comprising the nucleic acid as described above.

The present invention also provides a cell expressing the vector or the plasmid as described above.

The invention also provides a pharmaceutical composition, comprising the bispecific antibody as described above and at least one pharmaceutically acceptable excipient, diluent or carrier.

The present invention also provides use of the bispecific antibody as described above in the following, comprising: (a) preparing a drug for the treatment of an oncological disease comprising one or more of breast cancer, gastric cancer, colorectal cancer, membranous adenocarcinoma, lung cancer, oesophageal cancer, prostate cancer, connective tissue proliferative small round cell tumor, ovarian cancer, pancreatic cancer, liver cancer, renal cancer, non-small cell lung cancer, melanoma, alveolar rhabdomyosarcoma, embryonal rhabdomyosarcoma, Ewing's sarcoma, nephroblastoma, neuroblastoma, ganglion cell tumor, medulloblastoma, high-grade glioma, diffuse intrinsic pontine glioma or multi-layered chrysoidal mass embryonal tumors; or (b) preparing a reagent or a kit for the detection of B7H3 and/or NKP30 molecules.

The invention is further described below in connection with embodiments which are used to describe some specific embodiments of the invention and are not intended to limit the scope of protection of the invention. Unless otherwise defined, all technical and scientific terms used herein have the same meaning as those generally understood by ordinary technicians in the field to which this application belongs. Although methods and materials similar to or equivalent to the methods and materials described herein may be used in the practice or testing of this application, appropriate methods and materials are described below. In case of contradiction, the patent specification shall prevail.

The term “antibody” as used herein, is also referred to as “immunoglobulin” and can be a natural or conventional antibody in which the two heavy chains are linked to each other by disulfide bonds and each heavy chain is linked to a light chain by a disulfide bond. There are two types of light chains, λ(l) and κ(k), and five main heavy chain species (or isotypes), IgM, IgD, IgG, IgA and IgE, which determine the functional activity of the antibody molecule. Each chain comprises a different sequence of domains. The light chain consists of two domains, the variable domain (VL) and the constant domain (CL). The heavy chain consists of four domains, i.e. the variable heavy chain region (VH) and three constant regions (CH1, CH2 and CH3, collectively referred to as CH). Both the variable region of the light chain (VL) and the variable region of the heavy chain (VH) determine the specificity of the binding recognition of the antigen. The constant domain (CL) of the light chain and the constant region (CH) of the heavy chain have important biological roles such as antibody chain binding, secretion, complement binding and binding to the Fc receptor (FcR). The light and heavy chains of the antibody each have three CDRs, called LCDR1, LCDR2, LCDR3 and HCDR1, HCDR2, HCDR3 respectively. conventional antibody antigen binding sites thus comprise six CDRs comprising a collection of CDRs from each of the variable regions of the heavy and light chains. the CDRs can result in different sequences depending on the numbering system. The CDRs listed in the present invention are labelled using the Kabat numbering system.

The term “bispecific antibody” as used herein, refers to an antibody with binding specificity for at least two different epitopes. Bispecific antibody may be heterologous antibodies, for example they may be or may comprise two or more linked antibodies or antigen binding fragments (e.g. Fab), wherein each antibody or antigen binding fragment has a different binding specificity. In the present invention, “bispecific antibody”, “bispecific antigen binding proteins” and “bis-antibody” are used interchangeably.

The terms “monoclonal antibody” and “McAb” as used herein, refers to a highly homogeneous antibody produced by a single B-cell clone and directed only against a specific antigenic epitope. They are usually prepared using the hybridoma technique, which is based on a cell fusion technique in which sensitised B cells with the ability to secrete specific antibodies are fused with myeloma cells that have the ability to multiply indefinitely to form a B cell hybridoma. Single hybridoma cells with this characteristic are cultured into cell populations to produce specific antibodies against an antigenic epitope, i.e. monoclonal antibodies.

The term “VHH” as used herein, is also known as heavy chain single domain antibody, single domain antibody, VHH domain, VHH antibody fragment or VHH antibody, and refers to a single antigen binding polypeptide comprising only a heavy chain variable region. In some embodiments, VHH refers to a single domain antibody derived from a camel-derived antibody or a shark antibody with only one heavy chain variable region. In other embodiments, VHH refers to a single domain antibody derived from the heavy chain variable region (VH) of a monoclonal antibody, having only one heavy chain variable region.

The terms “polypeptide linker” and “linker” as used herein, have the same meaning and generally refer to a synthetic amino acid sequence that links or couples two peptide sequences (e.g. links two peptide domains). A polypeptide linker may link two amino acid sequences through a peptide bond. In some embodiments, the linker used in the present invention is (GGGGS)n, and when n=1, the linker corresponds to the amino acid sequence SEQ ID NO: 9.

The term “homology” as used herein, generally refers to sequence similarity or exchangeability between two or more polynucleotide sequences or between two or more polypeptide sequences. In some embodiments, homologous polynucleotides are those sequences that are hybridized under stringent conditions and have at least 80% (e.g. 80%, 81%, 82%, 83%, 84%, 85%, 86%, 87%, 88%, 89%, 90%, 91%, 92%, 93%, 94%, 95%, 96%, 97%, 98%, 99% or 100%) of sequence identity.

The term “vector” as used herein, generally refers to a nucleic acid molecule capable of self-replication in a suitable host, which transfers the inserted nucleic acid molecule into and/or between host cells. The term may include vectors primarily for insertion of DNA or RNA into a cell, vectors primarily for replication of DNA or RNA, and expression vectors for transcription and/or translation of DNA or RNA. It also comprises vectors that perform more than one of these functions. An “expression vector” is a polynucleotide that can be transcribed and translated into a polypeptide when introduced into a suitable host cell.

The term “isotype control” as used herein, refers to an antibody of the same subtype as the experimental antibody but not related to the target site.

### BRIEF DESCRIPTION OF THE DRAWINGS

FIG. 1 is a schematic diagram of the constructed antibody in the present invention.

FIG. 2 is another schematic diagram of the constructed antibody in the present invention.

FIG. 3 is an ELISA assay for the binding activity of the constructed antibody to B7H3 protein, wherein negative control is IgG1 isotype control; and positive control is B7H3 chimeric antibody.

FIG. 4 is an ELISA assay for the binding activity of the constructed antibody to NKP30 protein, wherein negative control is IgG1 isotype control; and positive control is NKP30 chimeric antibody.

FIG. 5 is an ELISA assay for the binding activity of the constructed antibody to B7H3 protein and NKP30 protein at both ends, wherein negative control is IgG1 isotype control.

FIG. 6 is the result of experiments in which the constructed antibody of the present invention activates NK cells and secretes IFN-γ, wherein the positive control is the NKP30 monoclonal antibody and the negative control is the IgG1 isotype control.

FIG. 7 is the result of the effect of the concentration of the constructed antibody of the present invention on the lysis rate of MCF-7 cells.

FIG. 8 is the result of the effect of the concentration of the constructed antibody of the present invention on the lysis rate of gastric cancer cells HS-746T, wherein the negative control is the IgG1 isotype control.

FIG. 9 is the result of the effect of the concentration of the constructed antibody on the lysis rate of breast cancer cells JIMT-1.

### DETAILED DESCRIPTION

### Example 1 Acquisition and Optimization of Nucleotide Sequences

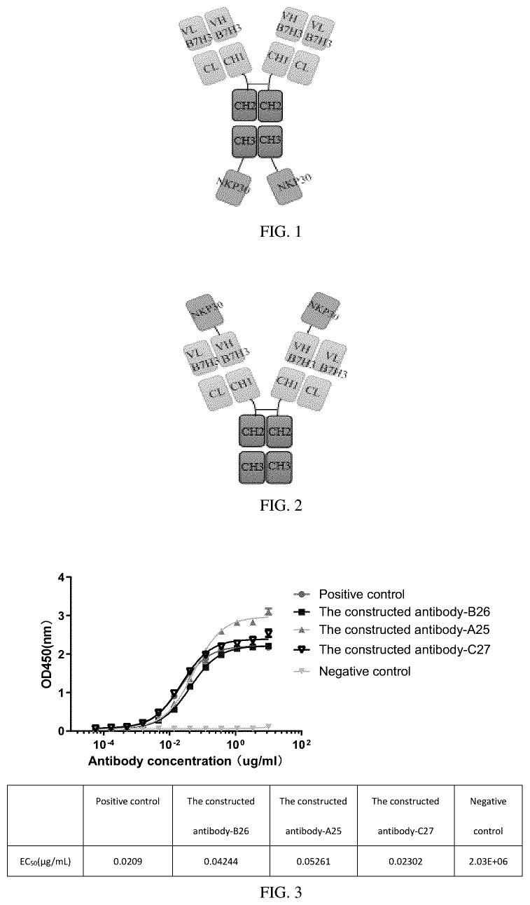

The constructed antibodies in the present invention are bispecific antibodies constructed using the B7H3 antibody and the NKP30 protein. The amino acid sequences of the light and heavy chains of the B7H3 antibody are derived from the sequence information of the publicly available B7H3 target monoclonal antibody, and the information on the variable and constant regions of the sequence is obtained by analysis (The amino acid sequence of the heavy chain variable region of the B7H3 antibody is shown in SEQ ID NO: 3; the amino acid sequence of the light chain variable region of the B7H3 antibody is shown in SEQ ID NO: 4, the amino acid sequence of the heavy chain constant region of IgG1 is shown in SEQ ID NO: 5, the amino acid sequence of the heavy chain constant region sequence of IgG4 is shown in SEQ ID NO: 6 and the amino acid sequence of the light chain constant region (as shown by SEQ ID NO: 7). NKP30 (The amino acid sequence is SEQ ID NO: 1 or SEQ ID NO: 2) is linked to any position on the light or the heavy chain of the B7H3 antibody by means of a linker. As required, the Fc of the amino acid sequence of the antibody is adjusted to other IgG types, such as IgG1, IgG4, etc., and further amino acid mutations of the desired form are designed in each heavy chain, resulting in the amino acid sequence of the target antibody. FIG. 1 and FIG. 2 list some of the structural schematics, and Table 1 shows the bispecific antibody combinations constructed from the example structural schematics, wherein the constructed antibodies A24, B23, A25 and B26 are shown in FIG. 1 and the structures of C27 and D28 are shown in FIG. 2.

TABLE 1
Amino acid sequence combinations for the constructed antibodies

| | Amino acid | Amino acid | | |
|---|---|---|---|---|
| | sequence of | sequence of | Amino acid | |
| | the first | the second | sequence of | |
| Constructed | polypeptide | polypeptide | VHH (NKP30 | |
| antibody | chain | chain | sequence) | Subtype |
| B23 | SEQ ID NO: 12 | SEQ ID NO: 11 | SEQ ID NO: 2 | IgG4 |
| A24 | SEQ ID NO: 13 | SEQ ID NO: 11 | SEQ ID NO: 2 | IgG1 |
| A25 | SEQ ID NO: 14 | SEQ ID NO: 11 | SEQ ID NO: 1 | IgG1 |
| B26 | SEQ ID NO: 15 | SEQ ID NO: 11 | SEQ ID NO: 1 | IgG4 |
| C27 | SEQ ID NO: 16 | SEQ ID NO: 11 | SEQ ID NO: 1 | IgG1 |
| D28 | SEQ ID NO: 17 | SEQ ID NO: 11 | SEQ ID NO: 1 | IgG4 |

### Example 2 Gene Synthesis and Construction of Expression Vectors

The pcDNA3.1-G418 vector was used as a dedicated vector for the expression of the light and heavy chains of the constructed antibody. The pcDNA3.1-G418 vector comprises the CMV Promoter used for the heavy chain, the eukaryotic screening marker G418 tag and the prokaryotic screening tag Ampicillin. In the construction of the expression vector, a signal peptide sequence (amino acid sequence as shown by SEQ ID NO: 8) was added to both the 5′ end of the heavy and light chains of the antibody. The nucleotide sequences of coding genes of the heavy chain, the light chain that the antibody expressed were obtained separately by gene synthesis, the vector and target fragments were double digested with HindIII and XhoI, recovered and then enzymatically linked by DNA ligase and transformed into E. coli receptor cells DH5a. Positive clones were selected and validated by plasmid extraction and enzymatic digestion to obtain recombinant plasmids containing coding genes of the heavy chain and the light chain of the antibody.

### Example 3 Plasmid Extraction

The recombinant plasmids containing each of the above target genes were transformed into E. coli receptor cells DH5a according to the method described in the Guide to Molecular Cloning Experiments (2002, Science Press), the transformed bacteria were coated on LB plates containing 100 μg/mL ampicillin, the plasmid clones were selected and cultured in liquid LB medium, shaken at 260 rpm for 14 h. The plasmids were extracted by an endotoxin-free plasmid macroextraction kit, lysed in sterile water and the concentrations were determined using a nucleic acid protein quantifier.

### Example 4 Plasmid Transfection, Transient Expression and Antibody Purification

ExpiCHO was incubated at 37° C., with 8% CO₂, at 100 rpm to a cell density of 6×10⁶ cells/mL. The constructed vector plasmids were transfected into the above cells at a mass concentration of 1:1 using liposomes, wherein the concentration of transfected plasmids was 1 mg/mL, liposome concentration was determined with reference to ExpiCHO™ Expression System kit, and were incubated at 32° C., 5% CO₂, and 100 rpm for 7-10 days. The product was supplemented after 18-22 h of transfection and on the 5th day once respectively. The above culture products were centrifuged at 4000 g in a centrifuge and filtered through a 0.22 μm filter membrane, and the medium supernatant was collected. The resulting antibody proteins were purified using ProteinA and ionic column, and the eluate was collected.

ProteinA and ionic column purification was performed as follows: cell culture broth was centrifuged at high speed and the supernatant was taken for affinity chromatography using GE's ProteinA chromatography column. The chromatography was performed using an equilibration buffer of 1×PBS (pH 7.4). The cell supernatant was combined and washed with PBS until UV was returned to baseline. Then the target protein was eluted using an elution buffer of 0.1 M glycine (pH 3.0), and stored at a neutral pH using Tris. The affinity chromatography product was adjusted to a pH 1-2 pH units below or above the isoelectric point pI and diluted appropriately to control the sample conductance below 5 ms/cm. NaCl gradient elution at the corresponding pH was carried out using a suitable corresponding pH buffer such as phosphate buffer, acetate buffer, etc., using the conventional ion exchange chromatography methods in the art such as anion exchange or cation exchange, and the collection tubes where the target proteins were located were combined and stored according to SDS-PAGE. The purified eluate was then ultrafiltered into a buffer.

### Example 5 Detection of Affinity of Antibody to B7H3 by ELISA

HuB7H3-his (purchased from ACROBiosystems) was diluted to 0.5 μg/mL using PBS buffer (pH 7.4) and added to a 96-well ELISA plate at 100 μL per well, and coated overnight at 4° C. After 1 hour closure with 1% BSA closure solution, the plate was washed three times with PBST, and the purified antibody was diluted to 10 μg/mL with 0.5% BSA sample diluent as the starting concentration and diluted in a 3 fold gradient for a total of 11 gradients, with an IgG1 isotype control was set up as a negative control and a B7H3 chimeric antibody (the heavy chain amino acid sequence is shown in SEQ ID NO: 10 and the light chain amino acid sequence is shown in SEQ ID NO: 11) was set up as a positive control, and was incubated at 100 μL per well for 1 h at 37° C. The plate was then washed 3 times with PBST and HRP-labelled goat anti-human IgGFc was diluted 1:20,000 with the sample diluent. 100 μL was added to each well and incubated for 1 h at room temperature. After washing the plate 4 times with PBST, 100 μL of TMB substrate was added to each well and incubated for 10 min at room temperature and protected from light. 100 μL of 1 M HCl was added to each well to terminate the colour development reaction. The absorbance value of each well in the 96-well plate was measured on a multifunctional enzyme standard at a wavelength of 450 nm and a reference wavelength of 570 nm, absorbance value (OD) per well=OD_{450 nm}−OD_{570 nm}. The concentrations of the antibodies were logarithmically plotted as the horizontal coordinate and the measured absorbance values per well as the vertical coordinate. Sigmoidal dose-response (Variable Slope) method (Graph Pad Prism Software, Graph Pad Software, San Diego, California) was chosen for non-linear regression to obtain the binding curve of the target antibody to B7H3 protein.

The results of ELISA for the binding of the constructs to B7H3 protein are shown in FIG. 3. The results show that the constructed antibodies show good binding activity to B7H3 protein at multiple concentrations with EC₅₀ of 0.02302-0.05261 μg/mL, which is not significantly different from the positive control and significantly better than the negative control, and the different antibody structures and subtypes all show high binding activity to B7H3 protein.

### Example 6 Detection of the Binding Activity of Antibodies to NKP30 by ELISA

The human-NKP30-His (purchased from ACRO Biosystems) was diluted to 0.5 μg/mL using pH 7.4 PBS buffer and 100 μL per well was added to a 96-well ELISA plate and coated overnight at 4° C. The plates were closed with 1% BSA blocking solution for 1 hour and washed 3 times with PBST. The constructed expression antibodies were diluted to 10 μg/mL with 0.5% BSA sample diluent as the starting concentration and subjected to a 3-fold gradient dilution for a total of 11 gradients, with a negative control (blank wells and IgG1 isotype control) and a positive control that is NKP30 monoclonal antibody (amino acid sequence as shown by SEQ ID NO: 18), 100 μL per well, and incubated for 1 h at 37° C. The plate was washed 3 times with PBST and HRP-labelled goat anti-human IgG Fc was diluted 1:20000 with sample diluent, 100 μL for each well was added and incubated for 1 h at room temperature. After washing the plate 4 times with PBST, 100 μL of TMB substrate was added into each well and incubated for 10 min at room temperature, avoiding light. Then 100 μL of 1 M HCl was added into each well to terminate the colour development reaction. The absorbance value of each well in the 96-well plate was measured on a multifunctional enzyme standard at a wavelength of 450 nm and a reference wavelength of 570 nm, absorbance value (OD) per well=OD_{450 nm}−OD_{570 nm}. The concentration of the constructed antibody was logarithmically used as the horizontal coordinate, the measured absorbance value per well as the vertical coordinate, and the Sigmoidal dose-response (Variable Slope) method (Graph Pad Prism Software, Graph Pad Software, San Diego, California) was selected for non-linear regression to obtain the binding curve between the target antibody and NKP30 protein.

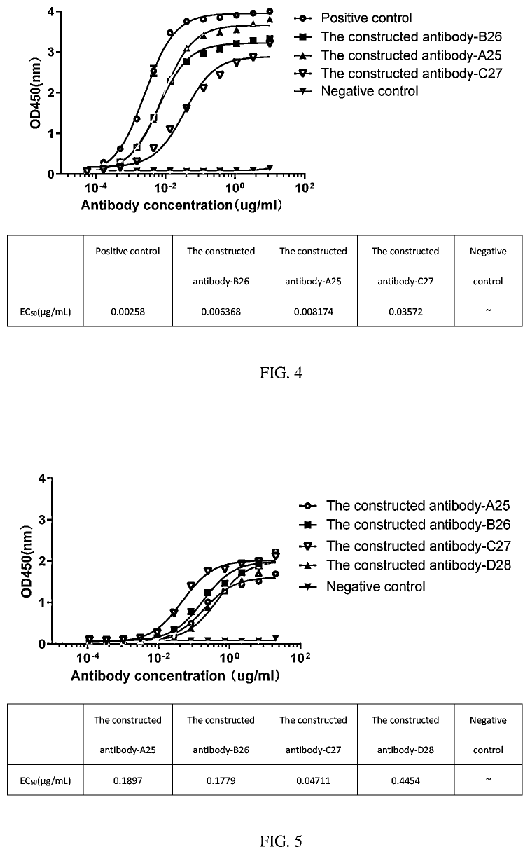

The ELISA results of the constructed antibodies are shown in FIG. 4. The constructed antibodies show good binding activity to NKP30 protein at multiple concentrations with EC₅₀ of 0.006368-0.03572 μg/mL, which is not significantly different from the positive control and significantly better than the negative control, and the different antibody structures and subtypes show high binding activity to NKP30 protein.

### Example 7 Binding Activity at Both Ends of the Constructed Antibody

HuB7H3-Fc (purchased from ACROBiosystems) was diluted to 0.5 μg/mL using PBS buffer (pH 7.4) and added to a 96-well ELISA plate at 100 μL per well, and coated overnight at 4° C. After 1 hour closure with 1% BSA closure solution, the plate was washed three times with PBST, and the purified antibody was diluted to 20 μg/mL with 0.5% BSA sample diluent as the starting concentration and diluted in a 3-fold gradient for a total of 11 gradients, and an IgG1 isotype control was set up as a negative control, and was incubated at 50 μL per well for 1 h at 37° C. The plate was then washed 3 times with PBST, and NKP30-his protein was diluted to 1 μg/mL, 100 μL of each well was added and incubated for 1 h at room temperature, then the plate was washed 3 times with PBST. The HRP-labelled his-antibody was diluted 1:20,000 with sample diluent, 100 μL was added to each well and incubated for 1 h at room temperature. After washing the plate 4 times with PBST, 100 μL of TMB substrate was added to each well and incubated for 10 min at room temperature and protected from light. 100 μL of 1 M HCl was added to each well to terminate the colour development reaction. The absorbance value of each well in the 96-well plate was measured on a multifunctional enzyme standard at a wavelength of 450 nm and a reference wavelength of 570 nm, absorbance value (OD) per well=OD_{450 nm}−OD_{570 nm}. The concentrations of the antibodies were logarithmically plotted as the horizontal coordinate and the measured absorbance values per well as the vertical coordinate. Sigmoidal dose-response (Variable Slope) method (Graph Pad Prism Software, Graph Pad Software, San Diego, California) was chosen for non-linear regression to obtain the binding curve of target antibodies to both ends of B7H3 and NKP30 proteins were obtained.

The ELISA results of the constructed antibodies are shown in FIG. 5. The negative control did not bind to both ends of B7H3 and NKP30 proteins, while the constructed antibodies with different structures and antibody subtypes show good binding activity to both ends of NKP30 and B7H3 proteins at all concentrations with EC₅₀ of 0.04711-0.4454 μg/mL. This result suggests that the binding of the constructed antibodies to B7H3 and NKP30 have little effect on each other, further suggesting that the constructed antibodies can bridge B7H3-expressing tumor cells to NK cells.

### Example 8 Activation of NK Cells by the Constructed Antibodies

The antibodies were diluted to 150 nM with PBS buffer, in 3-fold dilution, with a total of 8 gradients and 200 μL/well into a 96-well flat-bottom plate and incubated at 4° C. overnight. The incubated 96-well plate was taken and the antibody dilution was aspirated and then washed twice with PBS. The NK cells with medium containing IL-2 (400 U) were added, and 4×10⁴ cells with a total volume of 200 μL were added to the treated 96-well plates and incubated at 37° C. for 24 h. And then the cell culture supernatant was taken by centrifugation and the content of IFN-γ was detected.

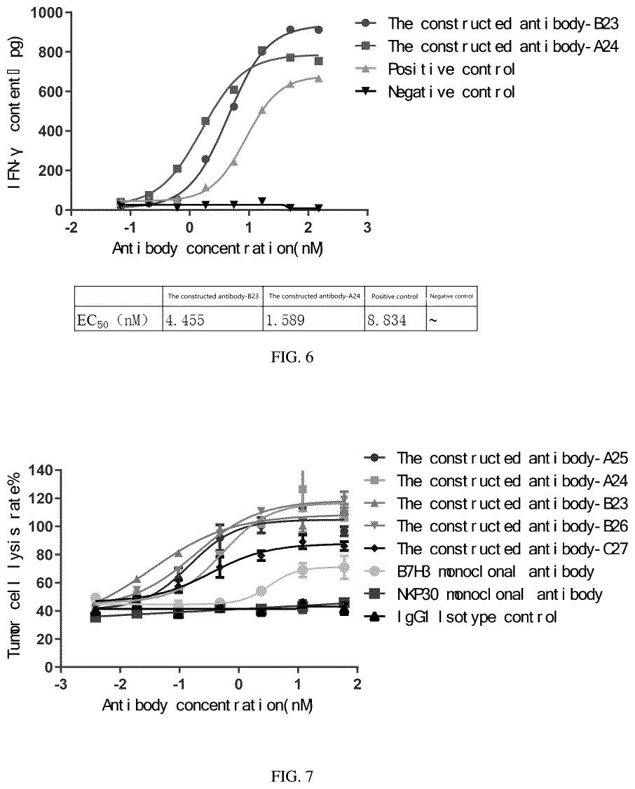

As shown from FIG. 6, the IFN-γ levels in the culture supernatant of NK cells stimulated by the antibodies are measured using a commercial IFN-γ cytokine assay kit in response to the activation of NK cells by the antibodies. The results show that NK cells stimulated with both NKP30 monoclonal antibody and the constructed antibodies secreted IFN-γ with an EC50 of 1.589 nM-8.834 nM, while the negative control stimulated NK cells at all concentrations did not produce IFN-γ, suggesting that both NKP30 monoclonal and constructed antibodies can specifically activate NK cell. The level of IFN-γ secretion from NK cells stimulated by the constructed antibodies is comparable to that of the NKP30 monoclonal control, suggesting that the constructed antibodies do not attenuate the activating NK cell activity of NKP30.

### Example 9 Assay for Specific Killing of B7H3-Positive Cells by NK Cells Mediated by the Constructed Antibodies

The constructed antibodies were selected to detect their specific killing assays against MCF-7 tumor cells expressing high B7H3, HS-746T tumor cells expressing high B7H3 and JIMT-1 tumor cells expressing high B7H3, respectively, and tumor cell mortality was detected by the LDH method (CytoTox96Non-RadioactiveCytotoxicity Assay, Cat: G1780).

A morphologically normal MCF-7 cell (or tumor cell HS-746T, or tumor cell JIMT-1) in log phase was used, first digested with trypsin and then neutralized using complete medium, then centrifuged at 1000 rpm for 4 min at room temperature and resuspended using RPMI1640 basal medium (containing 5% FBS), and then was spread in a 96-well plate at 2×10⁴/well, 50 μL/well. The constructed antibodies were diluted to 50 nM using RPMI1640 basal medium (containing 5% FBS), while it was later diluted in a 5-fold gradient, with a total of 8 concentration gradients and 100 μL/well, and set up in three replicates (the same experimental conditions, laying three wells). NK cells were resuspended and added to the corresponding wells at 6×10⁴/well and 50 μL/well to make the effect-target ratio 3:1. The target cell maximum lysis wells (M), target cell spontaneous release wells (ST), effector cell spontaneous release wells (SE), total volume-corrected blank wells (BV), and medium blank control wells (BM) were set up at the same time. After standing for 10 min, centrifugation was performed at 1000 rpm for 4 min at room temperature and incubated for 4 h in a 5% CO₂, and 37° C. carbon dioxide cell culture incubator. Lysate was added to wells M and B-V 45 min in advance, mixed well, and centrifuged at 1000 rpm for 4 min at room temperature at the end of incubation. 50 μL of supernatant was aspirated to the LDH analysis plate, 50 μL/well of substrate dissolved in analysis buffer (assay buffer) was added, and the reaction was performed for 30 min at room temperature and protected from light. 50 μL/well of termination solution was added and stood for 10 min, readings were taken at 490 nm. The cell lysis rate was calculated with the formulas OD (sample wells, ST, SE)-OD (B-M), OD (M)-OD (B-V), and cell lysis rate=OD (sample wells−ST-SE)×100/OD (M-ST), and the graph of the cell lysis rate and concentration-fitted data was plotted using GraphPadPrism software. The experimental results are shown in FIGS. 7, 8 and 9.

As shown from FIG. 7, both the negative control and NKP30 monoclonal antibody show no killing effect on tumor cells MCF-7, whereas the constructed antibodies and B7H3 monoclonal antibody show lytic death of MCF-7 cells in a concentration-dependent manner, suggesting that B7H3 monoclonal antibody and the constructed antibodies mediate the killing effect of NK cells on MCF-7 cells in a specific manner. The constructed antibodies at various concentrations show a higher rate of targeted tumor lysis on MCF-7 cells than B7H3 monoclonal antibody, suggesting that the constructed antibodies have higher targeted tumor lysis activity and that the constructed antibodies of all structures and subtypes could target tumor lysis. The constructed antibodies bind to both B7H3 and NKP30, bridging NK cells to target cells and allowing NK cells to produce closer immune synapses and kill target cells.

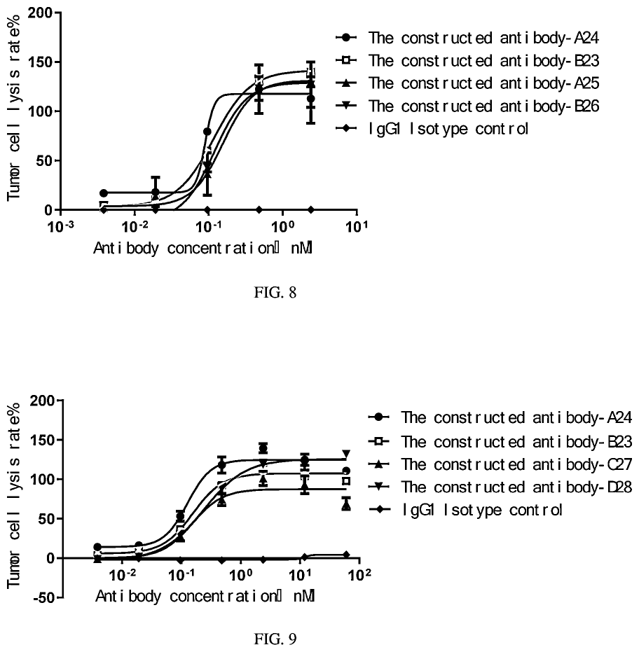

As shown from FIG. 8, the negative control has no targeted cleavage of tumor cell HS-746T activity, whereas the constructed antibodies show higher cleavage of tumor cell HS-746T activity in a concentration-dependent manner, suggesting that the constructed antibodies mediate the killing effect of NK cells on HS-746T cells in a specific manner.

As shown from FIG. 9, the negative control has no targeted cleavage of tumor cell JIMT-1 activity, whereas the constructed antibodies show higher cleavage of tumor cell JIMT-1 activity in a concentration-dependent manner, suggesting that the constructed antibodies mediate the killing effect of NK cells on JIMT-1 cells in a specific manner.

It should be noted that the above is only a preferred embodiment of the present invention and is not intended to limit the scope of the present invention. Any modifications, equivalent replacements and improvements etc. made within the spirit and principles of the present invention shall be included within the scope of protection of the present invention.

1. A bispecific antibody, comprising:
(a) a first antibody or antigen binding fragment thereof that specifically binds to a first antigen; and
(b) a second antibody or antigen binding fragment thereof that specifically binds to a second antigen;
wherein the first antigen is B7H3 and the second antigen is NKP30; or the first antigen is NKP30 and the second antigen is B7H3.

2. The bispecific antibody of claim 1, wherein,
the first antibody or antigen binding fragment thereof comprises a heavy chain and a light chain, and the second antibody or antigen binding fragment thereof comprises VHH; and
wherein the VHH is linked to the N-terminal or C-terminal end of the heavy or the light chain of the first antibody or antigen binding fragment thereof.

3. The bispecific antibody of claim 1, wherein the heavy chain variable region of one heavy chain of the first antibody or antigen binding fragment thereof forms an antigen binding site with one light chain variable region of the light chain, and the heavy chain variable region of the other heavy chain forms an antigen binding site with the light chain variable region of the other light chain.

4. The bispecific antibody of claim 2, wherein the bispecific antibody comprises one first antibody or antigen binding fragment thereof and one or more of VHH(s).

5. The bispecific antibody of claim 4, wherein the bispecific antibody comprises one first antibody or antigen binding fragment thereof and one VHH, and the VHH is linked to the N-terminal or C-terminal end of the heavy or the light chain of the first antibody or antigen binding fragment thereof.

6. The bispecific antibody of claim 4, wherein the bispecific antibody comprises one first antibody or antigen binding fragment thereof and two VHHs.

7. The bispecific antibody of claim 6, wherein two VHHs are linked to the N-terminal end of each of the two heavy or two light chains of the first antibody or antigen binding fragment thereof; or, two VHHs are linked to the C-terminal end of each of the two heavy or two light chains of the first antibody or antigen binding fragment thereof.

8. The bispecific antibody of claim 7, wherein the bispecific antibody comprises two first polypeptide chains and two second polypeptide chains, wherein
(a) the first polypeptide chain each independently comprises a heavy chain of the first antibody or antigen binding fragment thereof and the VHHs; and
(b) the second polypeptide chain each independently comprises a light chain of the first antibody or antigen binding fragment thereof.

9. The bispecific antibody of claim 7, wherein the bispecific antibody comprises two first polypeptide chains and two second polypeptide chains, wherein
(a) the first polypeptide chain each independently comprises a heavy chain of the first antibody or antigen binding fragment thereof; and
(b) the second polypeptide chain each independently comprises a light chain of the first antibody or antigen binding fragment thereof and the VHHs.

10. The bispecific antibody of claim 8, wherein two first polypeptide chains are the same or different, and/or two second polypeptide chains are the same or different.

11. The bispecific antibody of claim 10, wherein two VHHs are linked via a linker to the N-terminal or C-terminal ends of the two heavy chains or the two light chains of the first antibody or antigen binding fragment thereof, respectively.

12. The bispecific antibody of claim 11, wherein the linker has an amino acid sequence as shown by (G₄S)x, wherein x is an integer selected from 1-6; preferably, x is an integer selected from 1-3.

13. The bispecific antibody of claim 1, wherein the heavy chain of the first antibody or antigen binding fragment thereof comprises a heavy chain variable region and a heavy chain constant region, and the light chain comprises a light chain variable region and a light chain constant region; preferably, the first antibody or antigen binding fragment thereof is a full-length antibody.

14. The bispecific antibody of claim 1, wherein the heavy chain of the first antibody or antigen binding fragment thereof comprises a first Fc region and a second Fc region.

15. (canceled)

16. (canceled)

17. (canceled)

18. (canceled)

19. The bispecific antibody of claim 1, wherein the first antibody or antigen binding fragment thereof specifically binds to B7H3, and the VHH specifically binds to NKP30, wherein;
the first antibody or antigen binding fragment thereof comprises HCDR1 as shown by SEQ ID NO: 19, HCDR2 as shown by SEQ ID NO: 20, and HCDR3 as shown by SEQ ID NO: 21; and LCDR1 as shown by SEQ ID NO: 22, LCDR2 as shown by SEQ ID NO: 23, and LCDR3 as shown by SEQ ID NO: 24; and
HCDR1 of the VHH is selected from any of the amino acid sequences of SEQ ID NO: 25 or SEQ ID NO: 28, or is a sequence having at least 80% identity to any of the amino acid sequences of SEQ ID NO: 25 or SEQ ID NO: 28; HCDR2 is selected from any of the amino acid sequences of SEQ ID NO: 26 or SEQ ID NO: 29, or is a sequence having at least 80% identity to any of the amino acid sequences of SEQ ID NO: 26 or SEQ ID NO: 29; HCDR3 is selected from any of the amino acid sequences of SEQ ID NO: 27 or SEQ ID NO: 30, or is a sequence having at least 80% identity to any of the amino acid sequences of SEQ ID NO: 27 or SEQ ID NO: 30.

20. The bispecific antibody of claim 19, wherein the first antibody or antigen binding fragment thereof comprises a heavy chain variable region VH as shown by SEQ ID NO: 3, and a light chain variable region VL as shown by SEQ ID NO: 4, and
the heavy chain variable region VH of the VHH is selected from any of the amino acid sequences of SEQ ID NO: 1 or SEQ ID NO: 2, or is a sequence having at least 80% identity to any of the amino acid sequences of SEQ ID NO: 1 or SEQ ID NO: 2.

21. The bispecific antibody of claim 20, wherein the first antibody or antigen binding fragment thereof comprises:
a heavy chain variable region VH as shown by SEQ ID NO: 3, and a light chain variable region VL as shown by SEQ ID NO: 4, and
the VHH comprises:
(a) a heavy chain variable region VH as shown by SEQ ID NO: 1, or
(b) a heavy chain variable region VH as shown by SEQ ID NO: 2.

22. The bispecific antibody of claim 1, wherein the first polypeptide chain of the bispecific antibody is selected from any of the amino acid sequences of SEQ ID NO: 12, SEQ ID NO: 13, SEQ ID NO: 14, SEQ ID NO: 15, SEQ ID NO: 16 or SEQ ID NO: 17, or is a sequence having at least 80% identity to any of the amino acid sequences of SEQ ID NO: 12, SEQ ID NO: 13, SEQ ID NO: 14, SEQ ID NO: 15, SEQ ID NO: 16 or SEQ ID NO: 17; and the second polypeptide chain of the bispecific antibody is as shown by SEQ ID NO: 11, or is a sequence having at least 80% identity to SEQ ID NO: 11.

23. The bispecific antibody of claim 1, wherein the bispecific antibody comprises:
(1) the first polypeptide chain as shown by SEQ ID NO: 12, and the second polypeptide chain as shown by SEQ ID NO: 11;
(2) the first polypeptide chain as shown by SEQ ID NO: 13, and the second polypeptide chain as shown by SEQ ID NO: 11;
(3) the first polypeptide chain as shown by SEQ ID NO: 14, and the second polypeptide chain as shown by SEQ ID NO: 11;
(4) the first polypeptide chain as shown by SEQ ID NO: 15, and the second polypeptide chain as shown by SEQ ID NO: 11;
(5) the first polypeptide chain as shown by SEQ ID NO: 16, and the second polypeptide chain as shown by SEQ ID NO: 11; or
(6) the first polypeptide chain as shown by SEQ ID NO: 17, and the second polypeptide chain as shown by SEQ ID NO: 11.

24. (canceled)

25. (canceled)

26. (canceled)

27. (canceled)

28. (canceled)
